(19) United States
(12) Patent Application Publication
(10) Pub. No.: US 2012/0209231 A1
(43) Pub. Date: Aug. 16, 2012

(54) TEXTILES CONTAINING IMPROVED SUPERABSORBERS

(75) Inventors: Peter Rudolf, Ladenburg (DE); Andreas Brockmeyer, Alsbach-Hahnlein (DE)

(73) Assignee: BASF SE, Ludwigshafen (DE)

(21) Appl. No.: 13/503,984

(22) PCT Filed: Nov. 2, 2010
(86) PCT No.: PCT/EP10/66578

§ 371 Date: Apr. 25, 2012

(30) Foreign Application Priority Data
Nov. 6, 2009 (EP) 09175284.0

(51) Int. Cl. A61F 13/53; F26B 5/16; A47C 27/00; B32B 37/14; A47C 7/02; B32B 5/02; B32B 37/02

(52) U.S. Cl. 604/367; 442/118; 34/329; 156/60; 156/148; 297/452.48; 5/690

(57) ABSTRACT

Improved superabsorbent-containing textiles comprise hydrophilic fibers incorporated after application of superabsorbent. This makes it possible to provide superabsorbent on a relatively open-pore textile, but the distribution of liquid applied to the textile is distinctly improved by the hydrophilic fibers additionally incorporated.

The present invention provides improved superabsorbent-containing textiles, processes for their production, and their use for water absorption including the use for moisture regulation. The invention provides more particularly superabsorbent-containing textiles in which the distribution of liquid is improved.

Superabsorbent-containing textiles are known. In such textiles, the superabsorbent is a constituent part of the textile in that it is, for example, produced on the textile by chain growth addition polymerization of an appropriate monomeric solution or suspension applied to the textile, or is incorporated, as a ready-formed pulverulent or fibrous superabsorbent, in the textile in the course of the production thereof.

Superabsorbents themselves are likewise known. Other common terms for such materials are “high-swellability polymer”, “hydrogel” (often even used for the dry form), “hydrogel-forming polymer”, “water-absorbing polymer”, “absorbent gel-forming material”, “swellable resin”, “water-absorbing resin” and the like. Superabsorbents comprise crosslinked hydrophilic polymers, in particular polymers of (co)polymerized hydrophilic monomers, graft (co)polymers of one or more hydrophilic monomers on a suitable grafting base, crosslinked ethers of cellulose or of starch, crosslinked carboxymethyl-cellulose, partially crosslinked polyalkylene oxide or natural products swellable in aqueous fluids, examples being guar derivatives, although water-absorbing polymers based on partially neutralized acrylic acid are most common. The essential property of superabsorbents is their ability to absorb and retain amounts of aqueous fluids equivalent to many times their own weight, even under moderate pressure. As the dry superabsorbent takes up liquid, it turns into a gel, a hydrogel in the usual cases of the absorption of water. Their crosslinking distinguishes synthetic superabsorbents in an essential and important way from customary pure thickeners, since the crosslinking renders the polymers insoluble in water. Soluble substances would have no utility as superabsorbents. By far the most important field of use of superabsorbents is to absorb bodily fluids. Superabsorbents are used for example in diapers for infants, incontinence products for adults or feminine hygiene products. Other fields of use include for example those as a water-retaining agent in market gardening, as a water store for protection against fire, for fluid absorption in food packaging or, very generally, for absorption of moisture.

The state of the art of superabsorbents is summarized for example in the monograph “Modern Superabsorbent Polymer Technology”, F. L. Buchholz and A. T. Graham, Wiley-VCH, 1998, pages 69 to 117.

WO 01/56625, EP-A 1 178 149 and U.S. Pat. No. 5,962,068 describe processes for producing water-absorbing textiles wherein water-absorbing polymers are polymerized onto a backing or substrate material. WO 2006/106096 A1 describes moisture-regulating textiles comprising at least one sheetlike backing or substrate material, at least one water-soluble hygroscopic substance and at least one superabsorbent polymerized onto the backing or substrate material in the presence of the water-soluble hygroscopic substance. JP-A 05-105705 concerns nondeliquescent driers consisting of a backing or substrate material and hygroscopic salts wherein the hygroscopic salts are fixed to the backing or substrate material by means of superabsorbents. WO 2007/023085 A1 teaches moisture-regulating textiles which do not form any undesirable unevennesses on contact with relatively large amounts of liquid (for example when liquid is spilt onto the textile). The moisture-regulating textiles of WO 2007/023086 A1 comprise a plasticizer in order that undesirable stiffness may be avoided.

WO 00/64311 discloses textiles wherein superabsorbents were polymerized onto a backing or substrate material. The composites are used for moisture regulation in seat padding. WO 2004/067826 A1 teaches multilayered textile sheet materials, in particular those comprising one-sidedly stitchbonded nonwovens, which may comprise active components such as superabsorbents for example and are suitable as a padding or cushioning material. DE 40 01 207 A1, DE 40 34 920 A1, DE 41 27 337 A1, DE 42 06 895 A1, DE 197 26 810 C1 and DE 198 09 156 A1 relate to the use of moisture-regulating textiles in seat furniture, in particular in motor vehicle seats.

An ever-present problem with the use of superabsorbents is that of ensuring that the liquid to be absorbed is efficiently distributed over the available quantity of superabsorbent. This is particularly so in the case of hygiene articles, into which the liquid to be absorbed is introduced within a short time in a comparatively large amount and in a locally concentrated fashion, but can also be relevant in other applications if, for example, liquid is spilt on a climate-regulating layer of a seat pad. If, in response, superabsorbent swells only locally, a gel layer forms and blocks the continued ingress of liquid (the phenomenon is known as gel-blocking), and if the separations between the gel particles are too large, the liquid will pass through unabsorbed. Hygiene articles therefore usually integrate in their “absorbent core” (often also referred to as “diaper core” or just “core” liquid-distributing layers to ensure uniform distribution into a liquid-storing layer. This storage layer contains the entire or at least the predominant amount of superabsorbent which permanently absorbs the applied liquid. The superabsorbent in the storage layer is typically mixed with cellulose fibers (“fluff”) to ensure transportation of liquid within the storage layer.

U.S. Pat. No. 5,728,085 describes a hygiene article wherein cellulose pulp in roll form is used directly and without the otherwise customary further fluffing as absorbent layer.

US 2002/0 123 728 A1 teaches a liquid distribution layer for hygiene articles which combines crosslinked and noncrosslinked cellulosic fibers.

According to WO 2005/094 749 A2, the liquid distribution problem is solved by coupling a relatively low level of fluff with the use of a superabsorbent which itself has substantial flow productivity problems and is combined with hydrophilic dendritic polymer and water-insoluble phosphate.

U.S. Pat. No. 6,140,550 discloses a comparatively open, fibrous structure such as, for example, an open-cell polyurethane foam that contains superabsorbent. The open structure permits unhindered ingress of liquid. This structure can be laminated with further sheet materials such as, for example, webs of hydrophilic fibers. U.S. Pat. No. 5,451,452 combines a superabsorbent in foam form with a textile ply for liquid distribution.

According to WO 95/35 081 A1, a hygiene article's absorbent member, constructed essentially of fiber web or wadding (often referred to as “fluff”) and superabsorbent, has strips of denser material installed in it to achieve better distribution in the absorbent member of incoming liquid. WO 01/21 122 A1 teaches absorbent members wherein superabsorbents are disposed in concentrated form in longitudinal bands extending through a fluff matrix. The channeling resulting particularly after a first swelling of the superabsorbent furthers the distribution of liquid.

WO 97/40 223 A1 describes a process for producing a fibrous nonwoven web having different pore sizes. Such a structure exhibits superior liquid distribution.

WO 03/053 483 A1 teaches a hygiene article top sheet fabricated from hydrophobic material but having been durably hydrophilicized at the surface. This gives improved liquid distribution into the absorbent member underneath the top sheet.

US 2004/0 254 551 A1 discloses an absorbent core structure for hygiene articles which does not require liquid distribution layers and which combines fluff, superabsorbent, binding elements (bicomponent fibers for example) and thin hollow fibers.

WO 94/24 975 A1 teaches the use of thin hydrophilic fibers in the core, alongside fluff, to improve liquid distribution.

Superabsorbent-containing textiles present an additional problem. When such materials are produced by spraying the textile with a monomeric mixture and subsequent chain growth addition polymerization, firmly adherent particles of superabsorbent form, which is generally desirable. Additionally, however, it would be desirable for the textile to be relatively open-pore, since otherwise the monomeric mixture which has been sprayed onto it only remains on the surface of the textile and, following chain growth addition polymerization, forms a comparatively hard surface layer of superabsorbent having few gaps between the superabsorbent particles, which exhibits pronounced “gel-blocking”. As a result, the textile loses flexibility to a quite appreciable extent, which is undesirable in applications such as storage layer in hygiene articles or as moisture-regulating layer in pads. If, however, a very open-pore textile is used, applied liquid is often only unsatisfactorily absorbed, since the open-pore textile itself is not very good at intermediate storage and distribution of liquids. An appreciable portion of applied liquid then merely passes unabsorbed through the textile.

It is an object of the present invention to provide an improved superabsorbent-containing textile. More particularly, said textile should be better able than prior art textiles to distribute applied liquid within itself, so that this liquid can be absorbed as completely as possible by the superabsorbent present. In addition, the textile should lose very little of its flexibility and it should be possible to apply superabsorbent to it in an advantageous manner.

We have found that this object is achieved by an improved supersabsorbent-containing textile comprising hydrophilic fibers incorporated after application of superabsorbent.

The present invention further provides a process for producing it and also applications.

We have determined that the textile of the present invention and the process for producing it make it possible to provide a relatively open-pore textile with superabsorbent, but that liquid distribution is distinctly improved by the additionally incorporated hydrophilic fibers.

The textile can in principle be any kind of textile to which superabsorbent can be applied. Textiles are flexible fibrous assemblages, more particularly sheetlike flexible fibrous assemblages. Fibrous assemblages of this kind, in addition to fibers, include interstices (often also called “pores”) between the fibers. In the context of this invention, textiles are more particularly textile intermediate and end products such as slivers, fibrous webs (i.e., fibrous assemblages held together by the fibers' own adherence) or nonwovens (additionally consolidated fibrous webs), felts, (fuller's felts, needlefelts), wovens, bobbinets, braids, knits, scrims, lace, embroideries, stitchbondeds, tufteds, hybrid forms thereof, and also finished textile articles manufactured therefrom. The fundamental definitions of textiles and of other pertinent terminology in this field are laid down in German standard specification DIN 60000 (January 1969). In the context of this invention, open-cell sheetlike foams can also be used like textiles, and are comprehended by the term “textiles”. It is particularly for economic reasons that the application sectors in question here quite overwhelmingly use webs with or without additional consolidation beyond the fibers' own adherence, depending on the intended application. Webs are the textiles which are preferred for the present invention for economic reasons essentially. Any reference herein to “web” is therefore to be understood as synonymous for textiles. Materials such as the superabsorbent-containing textiles in question here are also often referred to simply as “superabsorbent webs” or “superab-sorbent-coated webs”.

The textile of the present invention comprises at least one textile as sheetlike backing material and at least one superabsorbent. It may comprise further constituents, in particular those already known as constituents of superabsorbent webs. Examples of such further constituents are hygroscopic substances or plasticizers.

Suitable webs for the present invention include those made using manufactured polymeric fibers. Manufactured polymeric fibers may be formed from any polymers capable of forming fibers which can be used to produce a web. Examples of suitable polymers are polyolefins such as polyethylene, polypropylene and the like, polyesters such as polyethylene terephthalate and the like, polyamides such as nylon-6, nylon-6,6, poly(iminocarboxypentamethylene) and the like, acrylics and modified cellulosic material such as cellulose acetate and rayon, and also mixtures and copolymers thereof.

Manufactured polymeric fibers may be formed by meltblowing, through a spunbond process, by extrusion and drawing, or other wet-, dry- and melt-spinning processes known to those skilled in the art. The manufactured polymeric fibers from which the web is formed may have a finite length or may be substantially continuous. For example, when manufactured polymeric fibers are formed by meltblowing, they can be substantially continuous (few visible ends). When fibers are formed by extrusion and drawing to produce a tow, the tow may be used as produced or cut into staple fibers having a length of, for example, about 25 millimeters to about 75 millimeters, or short cut into lengths of about 1 millimeter to about 25 millimeters. Manufactured polymeric fibers may suitably have a maximum cross-sectional dimension of about 0.5 micrometer to about 50 micrometers as determined by microscopic measurement using an optical microscope and a calibrated stage micrometer or by measurement from scanning electron micrographs.

The web may be formed directly through wet or dry laying, through a spunbond or meltblown process, for example by carding or air-laying staple or short-cut fibers. Other methods of forming webs known to those skilled in the art are also suited for use in the present invention. The web may subsequently be thermally or mechanically consolidated. Methods of consolidating webs are known to those skilled in the art and include thermal bonding, point bonding, powder bonding, ultrasonic bonding, chemical bonding, mechanical needling, waterjet bonding, stitching and the like.

The fibers may be homogeneous fibers or else multicomponent fibers, more particularly bicomponent fibers such as sheath/core or side-by-side fibers.

The web may be formed from a single type of manufactured polymeric fiber or may comprise manufactured polymeric fibers formed from different polymers, having different fiber lengths or fiber diameters. For example, the web may comprise a mixture of (1) bicomponent fibers having a polyethylene sheath and a polypropylene core, which bicomponent fibers have a maximum cross-sectional dimension of about 20 micrometers and a length of about 38 millimeters, and (2) polyester fibers (polyethylene terephthalate) having a maximum cross-sectional dimension of about 25 micrometers and a length of about 38 millimeters. Fibers 1 and 2 may be combined in a weight ratio of 1:99 to 99:1. The fibers may be uniformly mixed or may be concentrated at opposite planar surfaces of the web.

A suitable web consists in general of at least 10% by weight, preferably at least 20% by weight, more preferably at least 25% by weight and most preferably at least 50% by weight of manufactured polymeric fiber. The weight fraction of manufactured polymeric fiber may be 100% by weight. In addition to manufactured polymeric fibers, the web may comprise from 0% to 90% by weight of a nonmanufactured polymeric fiber such as wood pulp fluff, cotton linters, cotton and the like.

As a general rule, the polymers from which the manufactured polymeric fibers of the web are formed will be inherently hydrophobic. As used herein, the term “hydrophobic” describes a material which has a contact angle between water and the material of greater than 90 degrees. The term “hydrophilic” refers to a material where the contact angle between water and the material is less than 90 degrees. As used herein, a polymeric material will be considered to be “inherently hydrophobic or hydrophilic” when the polymeric material, free from any surface modifications or treatments (for example surfactants or spin finishes) is hydrophobic or hydrophilic, respectively.

The web generally has a basis weight of at least 20 g/m², preferably at least 30 g/m² and more preferably at least 50 g/m² and also generally of at most 800 g/m², preferably of at most 400 g/m² and more preferably of at most 200 g/m².

The web typically has a density of at least 0.005 g/cm³, preferably at least 0.008 g/cm³ and more preferably at least 0.01 g/cm³ and also generally of at most 0.12 g/cm³, preferably of at most 0.1 g/cm³ and more preferably of at most 0.08 g/cm³.

The web may further comprise hydrophilic fibers. The hydrophilic fibers may be inherently hydrophilic materials such as cellulosic fibers such as wood pulp fluff, cotton linters and the like; regenerated cellulose fibers such as rayon; or certain nylon copolymers such as poly(pentamethylenecarbonamide)-(nylon-6)/polyethylene oxide. Alternatively, hydrophilic fibers may be obtained from hydrophobic fibers by treatment with a hydrophilicizing agent. For example, hydrophilic fibers may be formed from a polyolefin which is subsequently coated with a surfactant such that the fiber itself becomes hydrophilic. Other methods of hydrophilicizing fibers formed from hydrophobic substances are likewise known and suited for use in the present invention.

Methods of providing inherently hydrophilic fibers such as wood pulp fluff are known, as are methods of forming regenerated cellulose fibers such as rayon or methods of hydrophilicizing hydrophobic fibers. When the hydrophilic fibers are obtained by hydro-philicization of hydrophobic fibers, the fibers will suitably have a fiber length and also a diameter within the ranges indicated above. When the hydrophilic fibers are inherently hydrophilic such as wood pulp fluff, rayon, cotton, cotton linters and the like, the fibers will generally have a length of about 1.0 millimeters to about 50 millimeters and a diameter of about 0.5 micrometers to about 100 micrometers.

The web may be formed from a single type of hydrophilic fiber or may comprise hydrophilic fibers having different compositions, lengths and diameters.

In one particular embodiment, the web consists of air-laid cellulosic fibers such as wood pulp fluff. Wood pulp fluff fibers are preferred owing to their ready availability and owing to the fact that they are relatively inexpensive compared to synthetic fibers. Such a web generally has a basis weight of at least 20 g/m², preferably at least 25 g/m² and more preferably of at least 50 g/m² and also generally of at most 200 g/m², preferably of at most 150 g/m² and more preferably of at most 125 g/m². The web typically has a density of at least 0.04 g/cm³, preferably at least 0.06 g/cm³ and more preferably of at least 0.08 g/cm³ and also generally of at most 0.20 g/cm³, preferably of at most 0.16 g/cm³ and more preferably of at most 0.14 g/cm³.

A further useful backing material in the superabsorbent web is one of the resilient textiles known, and often used, for composites in relation to seat furniture, mattress and motor vehicle seat covers. Commonly used resilient textiles are for example nonwovens of the Multiknit, Maliwatt, Malivlies or Kunit types. Such webs are manufactured by stitchbonding processes for example and are notable for partial reorientation of the usually longitudinal fibers in the transverse direction to thicken the web and create a certain resiliency or cushioning performance.

The superabsorbent web of the present invention comprises superabsorbent on or in the web used as backing material. The superabsorbent is for example formed on the web by polymerization of an appropriate monomer solution or suspension applied to the web, or is incorporated into the web as a ready-formed pulverulent or fibrous superabsorbent in the course of the production of the web, by producing the web in the presence of superabsorbent particles. In this case, any known superabsorbent can be used. The polymerization of a monomer solution applied to the web typically leads to superabsorbent particles particularly firmly adherent to the fibers and uniformly dispersed in the web, is also technically comparatively simple, and therefore is the preferred process for producing superabsorbent webs.

The monomer solution or suspension applied to the web (for example by spraying or by impregnating) for subsequent polymerization in this process typically comprises:

a) at least one ethylenically unsaturated acid-functional monomer which may be at least partly neutralized,

b) at least one crosslinker,

c) at least one initiator,

d) optionally one or more ethylenically unsaturated monomers copolymerizable with the monomers recited under a);

e) optionally one or more water-soluble polymers,

f) at least one solvent; and

g) optionally further additions and/or auxiliary materials.

The monomers a) are preferably water-soluble; i.e., the solubility in water at 23° C. is typically at least 1 g/100 g of water, preferably at least 5 g/100 g of water, more preferably at least 25 g/100 g of water and most preferably at least 35 g/100 g of water.

Suitable monomers a) are for example ethylenically unsaturated carboxylic acids, such as acrylic acid, methacrylic acid and itaconic acid. Acrylic acid and methacrylic acid are particularly preferred monomers. Acrylic acid is very particularly preferred.

Further suitable monomers a) are for example ethylenically unsaturated sulfonic acids, such as styrenesulfonic acid and 2-acrylamido-2-methylpropanesulfonic acid (AMPS).

Impurities can have an appreciable effect on the polymerization. Therefore, the raw materials used should be very pure. It is therefore often advantageous to specially purify the monomers a). Suitable methods of purification are described for example in WO 2002/055469 A1, WO 2003/078378 A1 and WO 2004/035514 A1. A suitable monomer a) is for example an acrylic acid purified as described in WO 2004/035514 A1 and comprising 99.8460% by weight of acrylic acid, 0.0950% by weight of acetic acid, 0.0332% by weight of water, 0.0203% by weight of propionic acid, 0.0001% by weight of furfurals, 0.0001% by weight of maleic anhydride, 0.0003% by weight of diacrylic acid and 0.0050% by weight of hydroquinone monomethyl ether.

The proportion of the total amount of the monomers a) which is attributable to acrylic acid and/or salts thereof is preferably at least 50 mol %, more preferably at least 90 mol % and most preferably at least 95 mol %.

Monomer a) is typically partly neutralized. True, it is theoretically possible to polymerize the monomer in the nonneutralized state and subsequently to neutralize the resulting polymeric gel, but in the case of superabsorbent webs an adequately homogeneous neutralization at that stage is usually costly and inconvenient and therefore uneconomical. Preferably, therefore, the monomer is partly neutralized. This is typically accomplished by admixing the neutralization agent as an aqueous solution, or else preferably as a solid, into the monomer or the monomer solution. The degree of neutralization of the monomer is usually at least 25 mol %, preferably at least 50 mol % and more preferably at least 60 mol % and also generally at most 95 mol %, preferably at most 80 mol % and more preferably at most 75 mol %. Customary neutralizing agents can be used, preference being given to alkali metal hydroxides, alkali metal oxides, alkali metal carbonates or alkali metal bicarbonates and also mixtures thereof. Ammonium salts can be used instead of alkali metal salts. Sodium and potassium are particularly preferred as alkali metals, but very particular preference is given to sodium hydroxide, sodium carbonate or sodium bicarbonate and also mixtures thereof.

The monomer solution is stabilized against premature polymerization with preferably up to 250 weight ppm, more preferably at most 130 weight ppm, even more preferably at most 70 weight ppm, more preferably at least 10 weight ppm, even more preferably at least 30 weight ppm and particularly with around 50 weight ppm of hydroquinone monoether, all based on the nonneutralized monomer a). For example, the monomer solution may be prepared using an ethylenically unsaturated acid-functional monomer comprising an appropriate level of hydroquinone monoether. This stabilizer is occasionally also referred to as “polymerization inhibitor” even though it is merely intended to inhibit an uncontrolled or premature polymerization and not the desired polymerization to form the superabsorbent.

Preferred hydroquinone monoethers are hydroquinone monomethyl ether (MEHQ) and/or alpha-tocopherol (vitamin E). These stabilizers require dissolved oxygen for optimum performance. Therefore, before polymerization, the monomer solution can be freed of dissolved oxygen by inertization, i.e., passing an inert gas, preferably nitrogen or carbon dioxide, through the monomer solution, and thereby the degree of stabilization of the monomer against polymerization be conveniently reduced. The level to which the oxygen content of the monomer solution is reduced before polymerization is preferably to less than 1 weight ppm, more preferably to less than 0.5 weight ppm and most preferably to less than 0.1 weight ppm.

Suitable crosslinkers b) are compounds having at least two groups suitable for crosslinking. Such groups are for example ethylenically unsaturated groups which can be free-radically polymerized into the polymer chain, and functional groups capable of forming covalent bonds with the acid groups of the monomer a). Useful crosslinkers b) further include polyvalent metal salts capable of forming coordinative bonds with at least two acid groups of monomer a).

Crosslinkers b) are preferably compounds having at least two polymerizable groups which can be free-radically polymerized into the polymer network. Suitable crosslinkers b) are for example ethylene glycol dimethacrylate, diethylene glycol diacrylate, polyethylene glycol diacrylate, allyl methacrylate, trimethylolpropane triacrylate, triallylamine, tetraallylammonium chloride, tetraallyloxyethane, as described in EP 530 438 A1, di- and triacrylates as described in EP 547 847 A1, EP 559 476 A1, EP 632 068 A1, WO 93/21237 A1, WO 2003/104299 A1, WO 2003/104300 A1, WO 2003/104301 A1 and DE 103 31 450 A1, mixed acrylates which, as well as acrylate groups, comprise further ethylenically unsaturated groups, as described in DE 103 31 456 A1 and DE 103 55 401 A1, or crosslinker mixtures as described for example in DE 195 43 368 A1, DE 196 46 484 A1, WO 90/15830 A1 and WO 2002/32962 A2.

Preferred crosslinkers b) are pentaerythritol triallyl ether, tetraalloxyethane, methylene-bismethacrylamide, 15-tuply ethoxylated trimethylolpropane triacrylate, polyethylene glycol diacrylate, trimethylolpropane triacrylate and triallylamine.

Very particularly preferred crosslinkers b) are the diacrylated, dimethacrylated, triacrylated or trimethacrylated multiply ethoxylated and/or propoxylated glycerols as described for example in WO 2003/104301 A1. Di- and/or triacrylates of 3- to 10-tuply ethoxylated glycerol are particularly advantageous. Very particular preference is given to di- or triacrylates of 1- to 5-tuply ethoxylated and/or propoxylated glycerol. The triacrylates of 3- to 5-tuply ethoxylated and/or propoxylated glycerol are most preferred, especially the triacrylate of 3-tuply ethoxylated glycerol.

The amount of crosslinker b) is generally in the range from 0.05% to 1.5% by weight, more preferably in the range from 0.1% to 1% by weight and most preferably in the range from 0.3 to 0.6% by weight, all based on monomer a).

As initiators c) it is possible to use any compound which forms free radicals under the polymerization conditions, for example thermal initiators, redox initiators or photoinitiators. Suitable redox initiators are sodium peroxodisulfate/ascorbic acid, hydrogen peroxide/ascorbic acid, sodium peroxodisulfate/sodium bisulfite and hydrogen peroxide/sodium bisulfite. Mixtures of thermal initiators and redox initiators are often used, such as sodium peroxodisulfate/hydrogen peroxide/ascorbic acid. As reducing component, however, it is preferable to use a mixture of the sodium salt of 2-hydroxy-2-sulfinatoacetic acid, the disodium salt of 2-hydroxy-2-sulfonatoacetic acid and sodium bisulfite. Such mixtures are available as Brüggolite® FF6 and Brüggolite® FF7 (Brüggemann Chemicals; Heilbronn; Germany). Superabsorbent webs are often also produced by photopolymerization, in which case suitable photoinitiators are used. Preferred initiators include water-soluble azo compounds such as 2,2′-azobis(2-(2-imidazol-2-yl))propane dihydrochloride and 2,2′-azobis(amidino)propane dihydrochloride, water-soluble benzophenones such as 4-benzoyl-N,N,N-trimethylbenzenemethanaminium chloride, 2-hydroxy-3-(4-benzoylphenoxy)-3-N,N,N-trimethyl-1-propanaminium chloride monohydrate, 2-hydroxy-3-(3,4-dimethyl-9-oxo-9H-thioaxanthon-2-yloxy)-N,N,N-trimethyl-1-propanaminiuni chloride, 2-hydroxy-1-[4-(hydroxyethoxy)phenyl]-2-methyl-1-propanone, 2-hydroxy-2-methyl-1-phenylpropan-1-one and 4-benzoyl-N,N-dimethyl-N-[2-(1-oxo-2-propenyl)oxy]ethylbenzenemethanaminium chloride. A particularly preferred initiator combination comprises not only an azo initiator but also 2-hydroxy-1-[4-(hydroxyethoxy)phenyl]-2-methyl-1-propanone.

The monomer solution or suspension comprises a sufficient amount of one or more initiators to fully polymerize the superabsorbent-forming monomer present in the monomer solution or suspension. The initiator quantity is typically in the range from 0.01% to 5.0% and preferably in the range from 0.2% to 2.0% by weight, based on the weight of monomer a).

Ethylenically unsaturated monomers d) copolymerizable with the ethylenically unsaturated acid-functional monomers a) are for example acrylamide, methacrylamide, hydroxyethyl acrylate, hydroxyethyl methacrylate, dimethylaminoethyl methacrylate, dimethylaminoethyl acrylate, dimethylaminopropyl acrylate, diethylaminopropyl acrylate, dimethylaminoethyl methacrylate, diethylaminoethyl methacrylate.

As water-soluble polymers e) there can be used polyvinyl alcohol, polyvinylpyrrolidone, starch, starch derivatives, modified cellulose, such as methylcellulose or hydroxyethylcellulose, gelatin, polyglycols or polyacrylic acids, preferably starch, starch derivatives and modified cellulose.

The monomer solution typically comprises a solvent or suspension medium f). Since it is mostly solutions which are used or suspensions comprising relatively small proportions of insoluble components (supersaturated solutions for example), only solutions will for simplicity be referenced hereinbelow. Any solvent or solvent mixture can be used that provides a satisfactory application of the monomer solution to the web. Water is mostly and preferably used. The water content of the monomer solution is generally at least 40% by weight, preferably at least 45% by weight and more preferably at least 50% by weight and also generally at most 75% by weight, preferably at most 70% by weight and more preferably at most 65% by weight. When the monomer solution is applied to the web by spraying, the water quantity is adjusted such that a readily sprayable solution is obtained. Alternatively, this can also be achieved by using thickeners. The viscosity to which the spraying solution is set is generally at least 20 centipoise, preferably at least 30 centipoise and more preferably at least 40 centipoise and also generally at most 400 centipoise, preferably at most 150 centipoise and more preferably at most 100 centipoise, all measured in a Brookfield viscometer. An increasing water content means increasing energy requirements at the subsequent drying and a decreasing water content may mean inadequate removal of the heat of polymerization.

The monomer solution optionally comprises further additions or auxiliary materials. Examples of such additions or auxiliary materials are hygroscopic substances, in particular sodium chloride, as described for example in WO 2006/106096 A1 or JP 05/105705 A, plasticizers, as described in WO 2007/023085 A1, thickeners or thickening materials, for example finely divided particulate superabsorbents as described in WO 01/56625 A2.

The order in which the components of the monomer solution are added to prepare the monomer solution is not particularly important as such, but for safety reasons it is preferable to add the initiator last.

A superabsorbent web is produced by first applying the monomer solution to the web used as backing material. Convenient methods of application involve spraying or dripping the monomer solution onto the web or impregnating the web with monomer solution, conveniently by passing a web through the monomer solution in a pad-mangle or comparable apparatus whereby the application of predetermined amounts of a liquid to a textile fabric is possible.

The monomer solution is typically applied in such amounts that the content obtained of ready-produced superabsorbent after final drying is generally at least 20 g/m², preferably at least 40 g/m² and more preferably at least 40 g/m² and also generally at most 700 g/m², preferably at most 500 g/m² and more preferably at most 400 g/m².

The monomer solution is preferably applied by spraying. Spraying can take place by means of any customary spraying device, for example through nozzles. Not only one-material nozzles but also two-material nozzles in which the monomer solution is nebulized by gas can be used. The gas used can be air or an inert gas such as nitrogen, argon or helium. Preference is given to use of air, nitrogen or of a nitrogen-air mixture. The use of an inert gas such as nitrogen has the advantage of promoting the removal of oxygen from the monomer solution and of thereby reducing the polymerization-inhibiting effect of stabilizers such as MEHQ.

After the monomer solution has been applied to the web, the web is subjected to conditions at which the monomers polymerize. Depending on the initiator in the monomer solution, these conditions comprise for example the action of heat, ultraviolet rays, electron beam rays or their combination on the web with the applied monomer solution. The polymerization can be carried out batchwise or continuously, for example by passing the web with the applied monomer solution through irradiation or heating sectors on a conveyor belt.

When the polymerization is initiated thermally, the reaction apparatus is not subject to any special limitations. In the case of batch polymerizations, the monomer solution on the web can be polymerized in an oven in air or an inert atmosphere or else in vacuo. In the case of a continuous polymerization, the web passes through a dryer, for example an infrared dryer, a through-air dryer or the like. The polymerization temperature is chosen as a function of the thickness of the substrate, the monomer concentration and the identity and amount of the thermal initiator used in the monomer solution such that complete polymerization is obtained apart from the residual monomer concentration tolerable in an individual case. Thermal polymerization temperature is typically in the temperature range from 20° C. to 150° C. and preferably from 40° C. to 100° C. The polymerization time depends on the polymerization temperature, but is typically in the range from a few seconds to 2 hours and preferably in the range from a few seconds to 10 minutes.

When the polymerization is initiated by means of ultraviolet radiation, conventional UV lamps are typically used. The irradiation conditions, such as the irradiation intensity and time, depend on the type of fiber substrate used, on the amount of monomer applied to the substrate and on the initiator quantity and type, and are chosen as is customary in the art. Irradiation is typically carried out using a UV lamp having an intensity in the range from 100 to 700 watts per inch, preferably in the range from 400 to 600 watts per inch, at a distance between 2 to 30 centimeters between UV lamp and substrate, for a period ranging from 0.1 seconds to 10 minutes. Irradiation with ultraviolet rays can take place in vacuo, in the presence of an inert gas, such as nitrogen, argon, helium or the like, or in air. Irradiation temperature is not critical in that the irradiation of the sprayed web can mostly be carried out at room temperature with satisfactory results.

Polymerization initiation by means of electron beams can be accomplished using for example a commercially available electron beam accelerator such as the Electrocurtain® C B 175 (Energy Sciences, Inc., Wilmington, Mass.). Accelerators operating in the 150 to 300 kilovolt range are acceptable. The beam current of such systems, typically in the range from 1 to 10 milliamperes, can be adjusted to obtain the desired dose of ionizing radiation. The ionizing radiation dose employed will vary somewhat, depending on factors such as the presence or absence of crosslinking monomers, the desired degree of polymerization for the polymer, the degree of crosslinking desired and the like. In general, it is desirable to irradiate the coated web with doses from about 1 to 16 megarads and preferably 2 to 8 megarads. Particularly when using lower doses is it desirable to purge oxygen from the monomer solution, for example by bubbling nitrogen through the solution before applying it to the web. The dose is preferably so chosen that no fiber degradation occurs.

After polymerization, the superabsorbent web is customarily dried, for example by drying in a forced air oven, passing through a hot air dryer, passing through a sector illuminated by infrared lamps, or other suitable and known measures and apparatus for drying fabric webs. Drying is continued until the desired moisture content is achieved for the superabsorbent.

The web used as backing material can be coated with monomer solution and irradiated on either or both of the sides.

If desired, the superabsorbent web can be aftertreated. Examples of possible after-treatments are the application of plasticizers, softeners, surfactants, other textile auxiliaries, the setting of a desired moisture content or the surface postcrosslinking (often also only “postcrosslinking”) of the superabsorbent particles. These measures can also be combined.

Suitable surface postcrosslinkers are compounds comprising groups capable of forming covalent bonds with two or more carboxylate groups on the polymer particles. Suitable compounds are for example polyfunctional amines, polyfunctional amidoamines, polyfunctional epoxides as described in EP 83 022 A2, EP 543 303 A1 and EP 937 736 A2, di- or polyfunctional alcohols as described in DE 33 14 019 A1, DE 35 23 617 A1 and EP 450 922 A2, or p-hydroxyalkylamides as described in DE 102 04 938 A1 and U.S. Pat. No. 6,239,230. Furthermore, DE 40 20 780 C1 describes cyclic carbonates, DE 198 07 502 A1 2-oxazolidone and its derivatives, such as 2-hydroxyethyl-2-oxazolidone, DE 198 07 992 C1 bis- and poly-2-oxazolidinones, DE 198 54 573 A1 2-oxotetrahydro-1,3-oxazine and its derivatives, DE 198 54 574 A1 N-acyl-2-oxazolidones, DE 102 04 937 A1 cyclic ureas, DE 103 34 584 A1 bicyclic amide acetals, EP 1 199 327 A2 oxetans and cyclic ureas and WO 2003/31482 A1 morpholine-2,3-dione and its derivatives as suitable postcrosslinkers. Preferred postcrosslinkers are ethylene carbonate, ethylene glycol diglycidyl ether, reaction products of polyamides with epichlorohydrin and mixtures of propylene glycol and 1,4-butanediol. Very particularly preferred postcrosslinkers are 2-hydroxyethyloxazolidin-2-one, oxazolidin-2-one and 1,3-propanediol. It is further possible to use postcrosslinkers which comprise additional polymerizable ethylenically unsaturated groups, as described in DE 37 13 601 A1.

When postcrosslinking is carried out, the amount of postcrosslinker will generally be in the range from 0.001% to 2% by weight, preferably in the range from 0.02% to 1% by weight and more preferably in the range from 0.05% to 0.2% by weight, all based on the amount of superabsorbent in the web.

In one further embodiment of the present invention, polyvalent cations are applied to the particle surface in addition to the postcrosslinkers, or as postcrosslinkers, before, during or after postcrosslinking. Useful polyvalent cations for the process of the present invention include for example bivalent cations, such as the cations of zinc, magnesium, calcium, iron and strontium, tervalent cations, such as the cations of aluminum, iron, chromium, rare earths and manganese, quadrivalent cations, such as the cations of titanium and zirconium. Useful counterions include chloride, bromide, sulfate, hydrogensulfate, carbonate, bicarbonate, nitrate, phosphate, hydrogenphosphate, dihydrophosphate, and carboxylate, such as acetate and lactate. Aluminum sulfate is preferred. Polyamines can also be used as polyvalent cations as well as metal salts.

The amount of polyvalent cation used is for example in the range from 0.001% to 1.5% by weight, preferably in the range from 0.005% to 1% by weight and more preferably in the range from 0.02% to 0.8% by weight, all based on the polymer particles.

Postcrosslinking is typically carried out by spraying a solution of the postcrosslinker onto the dried superabsorbent web. Drying is carried out after spraying, and the postcrosslinking reaction can take place not only before but also during drying. Spraying (application by impregnation is also possible in principle) and drying are carried out as described above for the polymerization of the monomer solution.

The postcrosslinkers are typically used in the form of an aqueous solution. The depth of penetration of the postcrosslinker into the superabsorbent particles can be controlled via the level of nonaqueous solvent or overall solvent quantity. When water is exclusively used as solvent, it is advantageous to add a surfactant. This improves wetting and reduces clumping. Preferably, however, solvent mixtures are used, for example isopropanol-water, 1,3-propanediol-water and propylene glycol-water, the weight mixing ratio preferably being in the range from 20:80 to 40:60.

After the superabsorbent has been applied, the superabsorbent web thus produced has hydrophilic fibers incorporated into it. In contrast to fibers, including hydrophilic ones, which were already a constituent part of the web at the time the superabsorbent was applied, these subsequently incorporated fibers do not bear any particles of su-perabsorbent. They merely serve to fill up the relatively large pores in the original superabsorbent web in order that the intermediate storage and distribution of liquid may be improved as a result.

Useful hydrophilic fibers include all the abovementioned inherently hydrophilic or hydrophilicized fibers which can also already be a constituent part of the web.

Examples of useful inherently hydrophilic or hydrophilicized fiber materials for the present invention are monocomponent fibers such as fibers of polyethylene, polypropylene, nylon-6, nylon-6,6, nylon-12, copolyamide, polyesters such as for example polyethylene terephthalate (PET), polyethylene terephthalate copolymers or mixtures thereof, or bicomponent fibers such as fibers of polypropylene/polyethylene terephthalate, polyethylene/PET, polypropylene/nylon-6; nylon-6/PET, polytrimethylene terephthalate; polyethylene terephthalate, polytetramethylene terephthalate; copolyester/PET, copolyester/nylon-6, copolyester/nylon-6,6, poly-4-methyl-1-pentene/PET, poly-4-methyl-1-pentene/nylon-6, poly-4-methyl-1-pentene/nylon-6; poly-4-methyl-1-pentene/nylon-6,6; PET/polyethylene naphthalate (PEN), nylon-6,6/poly-1,4-cyclohexane dimethyl (PCT), polypropylene/polybutylene terephthalate (PBI); nylon-6/copolyamide, polylactic acid/polystyrene, polyurethane/acetal or soluble copolyester/polyethylene.

Further examples of suitable fiber materials are cellulose and cellulose derivatives such as wood- or cotton-derived cellulose, cellulosic pulp, cellulose acetate, wood fibers, polyvinyl alcohol or polyacrylate.

Preference is given to using cellulosic or polyester fibers.

The hydrophilic fibers are generally from about 1.0 millimeter to about 50 millimeters in length and from about 0.5 micrometer to about 100 micrometers in diameter.

The hydrophilic fibers can be incorporated in the superabsorbent web using any method known for incorporating fibers in a web. Such methods are known from felt manufacture in particular. A very simple method is for sprinkled fibers to be incorporated by fulling, another is the needling of a superabsorbent web with sprinkled-on hydrophilic fibers. This can be done from one side of the web, but also from both sides.

The textile of the present invention can additionally be aftertreated using any known measure. It can, particularly for use in moisture regulation, be laminated with a face material and also, in addition to face material and superabsorbent web, comprise further layers. These are chosen according to the intended use. Examples of possible further layers are, for example, a texture-conferring layer underneath an artificial leather face material, spacer knits and flame-laminated foams, a rear-sided textile protective or reinforcing layer, or a water-impermeable rear-sided layer.

The textiles of the present invention are supremely useful for absorbing liquids, for example in hygiene articles, but also for moisture regulation, in particular in mattresses and seat pads, for example in seat furniture or automotive seats, and also in other interior trim or foot mats. Hygiene articles, seat pads or mattresses comprising at least one textile of the present invention have excellent absorption and regulation ability for moisture.

1. A superabsorbent-containing textile comprising hydrophilic fibers incorporated after application of a superabsorbent.

2. The textile according to claim 1 as a web.

3. The textile according to claim 1 as a polyester fiber web.

4. The textile according to claim 1 wherein the hydrophilic fibers are cellulose fibers.

5. The textile according to claim 1 wherein the superabsorbent is a crosslinked polymer based on partially neutralized acrylic acid.

6. A process for producing a textile defined in claim 1, comprising the steps of
i) applying a monomeric mixture to a textile,
ii) polymerizing the monomeric mixture to form a superabsorbent, and
iii) incorporating hydrophilic fibers.

7. The process according to claim 6 wherein the monomeric mixture comprises partially neutralized acrylic acid and a crosslinker.

8. The process according to claim 6 wherein the acrylic acid is at least 25 mol % neutralized.

9. The process according to claim 6 wherein hydrophilic fibers are incorporated in the textile by fulling or needling.

10. (canceled)

11. (canceled)

12. A hygiene article, seat pad or mattress comprising at least one textile according to claim 1.

13. A method of absorbing a liquid comprising contacting the liquid with a textile of claim 1.

14. A method of regulating moisture in an article comprising incorporating a textile of claim 1 in the article.
